(19) United States
(12) Patent Application Publication
(10) Pub. No.: US 2026/0144866 A1
(43) Pub. Date: May 28, 2026

(54) METHODS, DEVICES, AND SYSTEMS FOR PREDICTING TREATMENT RESPONSIVENESS OF CLEAR CELL RENAL CELL CARCINOMA PATIENTS

(71) Applicant: THE REGENTS OF THE UNIVERSITY OF CALIFORNIA, Oakland, CA (US)

(72) Inventors: Hang CHANG, Moraga, CA (US); Jian-Hua MAO, Moraga, CA (US); April W. MAO, Berkeley, CA (US); Shuchi GULATI, Davis, CA (US); Kenneth ICZKOWSKI, Davis, CA (US)

(21) Appl. No.: 19/395,282

(22) Filed: Nov. 20, 2025

Related U.S. Application Data
(60) Provisional application No. 63/723,639, filed on Nov. 22, 2024.

(51) Int. Cl. A61K 39/395; A61K 31/404; A61K 31/436; A61K 31/4439; A61K 31/47; A61K 31/506; C07K 16/28; G16H 50/20

(52) U.S. Cl. CPC A61K 39/3955; A61K 31/404; A61K 31/436; A61K 31/4439; A61K 31/47; A61K 31/506; C07K 16/2818; C07K 16/2827; G16H 50/20

(57) ABSTRACT

The present invention provides for a method for predicting whether a subject with clear cell renal cell carcinoma (ccRCC) will be responsive to a targeted therapy or immunotherapy. The present invention also provides for a system using machine learning for determining whether a subject with clear cell renal cell carcinoma (ccRCC) will be responsive to a targeted therapy or immunotherapy.

### CROSS-REFERENCE TO RELATED APPLICATIONS

This application claims priority to U.S. Provisional Patent Application Ser. No. 63/723,639, filed Nov. 22, 2024, which are hereby incorporated by reference.

### STATEMENT OF GOVERNMENTAL SUPPORT

The invention described and claimed herein was made utilizing funds supplied by the U.S. Department of Energy under Contract No. DE-AC02-05CH11231, U.S. Department of Defense under Grant BCRP, No. BC190820, and the National Institutes of Health (NIH) under Grant Nos. R01CA184476, R01CA249882, and K08CA273542. The government has certain rights in this invention.

### FIELD OF THE INVENTION

The present invention is in the field of renal cell cancer.

### BACKGROUND OF THE INVENTION

Renal cell carcinoma (RCC), one of the ten most common malignancies, is a group of heterogeneous tumors originating in the nephron. The most common type of renal cell carcinoma (RCC) is clear cell renal cell carcinoma (ccRCC), which accounts for approximately 75% of all renal cell carcinomas. Basically, the 5-year survival rate for patients with ccRCC is about 50-69 percent. While it drops sharply to nearly 10 percent as ccRCC grows large enough or develops distant metastasis.

ccRCC originates from the proximal tubules of the renal cortex and grows in an expansive manner. Extensive histopathological studies of ccRCC have been performed [1, 2]; and molecular insights were recently introduced [3] to re-classified each RCC into molecular subtypes with demonstrated relevance to clinical outcome. Thus, we wonder whether ccRCC could be subtyped with clinical significance based whole slide image (WSI) and any cellular morphometric biomarkers could be extracted to applied to clinical practices.

We recently developed a framework powered by artificial intelligence (AI) technique for the cellular morphometric biomarker (CMB) and cellular morphometric subtype (CMS) discovery. We analyzed lower grade gliomas and found CMS associated with specific molecular alterations, immune microenvironment and prognosis [4]. In the present study, we used this framework to identify CMB and CMS in ccRCC, and to evaluate their clinical impacts and underlying molecular annotation.

### SUMMARY OF THE INVENTION

The present invention provides for a method for predicting whether a subject with clear cell renal cell carcinoma (ccRCC) will be responsive to a targeted therapy or immunotherapy The present invention also provides for a system using machine learning for determining whether a subject with clear cell renal cell carcinoma (ccRCC) will be responsive to a targeted therapy or immunotherapy.

The present invention provides for a method for predicting whether a subject with clear cell renal cell carcinoma (ccRCC) will be responsive to targeted therapy or immunotherapy will be efficacious for treating a subject with ccRCC, comprising: (a) obtaining a ccRCC tumor sample from a subject suffering from ccRCC, or suspected thereof; (b) determining or measuring the value or quantity of one or more cellular morphometric biomarkers (CMB); (c) correlating the value or quantity determined or measured for the CMBs to determine whether the subject would benefit from a targeted therapy or immunotherapy; and (d) optionally treating the subject with the targeted therapy or immunotherapy.

The present invention provides for a method for predicting whether a subject with clear cell renal cell carcinoma (ccRCC) will be responsive to targeted therapy or immunotherapy will be efficacious for treating a subject with ccRCC, comprising: (a) obtaining a ccRCC tumor sample from a subject suffering from ccRCC, or suspected thereof; (b) determining or measuring the value or quantity of one or more cellular morphometric biomarkers (CMB); (c) calculating a Cellular Morphometric Biomarker Risk Score (CMBRS) using the CMBs determined or measured in step (b) using the formula:

$$CMBRS = {\sum }_{i = 1}^{N} ( coefficient of {CMB\_Category}^{i} ) * ( {CMB\_Category}^{i} ) ;$$

(d) correlating the CMBRS to a corresponding CMB risk group (CMBRG) to identity whether the subject is of a CMBRG low group or CMBRG high group; and (e) optionally treating the subject wherein a patient belonging to the CMBRG low group is treated with targeted therapy, while a patient belonging to the CMBRG high group is treated with immunotherapy.

In some embodiments, the CMBRS is calculated using the means described herein. In some embodiments, the CMBs comprise the CMBs described in Example 1 herein. In some embodiments, the determining or measuring step comprises the value or quantity of the CMBs described in Example 1.

Renal cell carcinoma (RCC), the most common type of kidney cancer, is a group of heterogeneous tumors originating in the nephron. The most common subtype of RCC is clear cell renal cell carcinoma (ccRCC), which accounts for approximately 75% of all RCC, and is a heterogeneous disease in the form of histopathology, molecular mechanism and clinical outcome. In some embodiments, the method comprises using a 73 cellular morphometric biomarker (CMB) signature, determining the corresponding CMB risk score (CMBRS) and CMB risk group (CMBRG) for the prediction of prognosis and treatment response in ccRCC patients. The prognostic and predictive value of CMBRS and CMBRG have been validated in additional public and hospital cohorts. In addition, the molecular annotation of CMBRG using genomic and proteomic data provides the potential mechanism of the clinical significance of CMBRG. Example 1 herein establishes and validates an accurate and robust patient stratification with significant association with underly molecular and biological mechanisms and clinical outcomes in ccRCC.

In some embodiments, the method comprises using AI technology to discover biomarkers from whole slide images of hematoxylin and eosin (H&E)-stained slides in ccRCC patients. The biomarkers are predictable of prognosis and drug response in ccRCC patients. CMBs are rapid, low-cost and robust biomarkers. Prior to this present invention, no effective biomarkers for ccRCC have been demonstrated for immunotherapy and mTOR inhibitors. The CMBs of the present invention provide the first accurate stratification to select patient for these treatment.

The present invention provides for a system using machine learning for determining the CMRS, which is described herein.

In some embodiments, the targeted therapy comprises administering a tyrosine kinase inhibitor (TKI), mTOR inhibitor, immune checkpoint inhibitors, or a combination therapy to the subject. The tyrosine kinase inhibitor (TKI) inhibits the signals that tumors use to grow. In some embodiments, the tyrosine kinase inhibitor (TKI) is sunitinib, pazopanib, axitinib, or cabozantinib inhibit the signals that tumors use to grow. The mTOR inhibitor blocks signals in cancer cells to inhibit their growth. In some embodiments, the mTOR inhibitor is everolimus or temsirolimus. The immune checkpoint inhibitor boosts the body's immune system to help it fight cancer cells. In some embodiments, the immune checkpoint inhibitor is Pembrolizumab, nivolumab, or atezolizumab. The combination therapy is a combination of immune checkpoint inhibitors and TKIs and is used for advanced ccRCC. In some embodiments, the combination therapy is nivolumab plus ipilimumab or pembrolizumab plus axitinib.) is increasingly being used for advanced ccRCC. The use of everolimus is described by Ryan et al. (“Adjuvant everolimus after surgery for renal cell carcinoma (EVEREST): a double-blind, placebo-controlled, randomized, phase 3 trial”, Lancet 402(10407):1043-1051, 2023), hereby incorporated by reference.

In some embodiments, the everolimus is administered as follows: everolimus 10 mg orally once daily administered for 54 weeks. Then interruptions and dose reductions for toxicity were permitted as stipulated in the protocol. Criteria for early removal from treatment included disease recurrence or symptomatic deterioration, unacceptable toxicity, delay in protocol treatment for greater than 28 days, or patient treatment refusal and/or withdrawal of consent. To monitor for recurrence, patients underwent physical exam and scans of the chest, abdomen, and pelvis every 18 weeks for the first year, every 6 months for two years, and then annually until recurrence, death, or a maximum of 10 years after randomization.

In some embodiments, histologic confirmation of either clear cell or non-clear cell RCC was required (collecting duct or medullary carcinomas excluded). Patients must have been considered either intermediate-high (no nodal metastases and tumor stage 1b with grade 3 or 4, tumor stage 2 with any grade, or tumor stage 3a with grade 1 or 2) or very-high risk (tumor stage 3a with grade 3 or 4, tumor stage 3b, 3c, or 4 with any grade, or nodal metastases with any tumor stage and any grade) for recurrence.

In some embodiments, the immunotherapy therapy is a cytokine therapy or immune checkpoint inhibitor therapy. In some embodiments, the immune checkpoint inhibitor therapy comprises administering an pembrolizumab, nivolumab, and/or atezolizumab to the subject.

In some embodiments, suitable mTOR inhibitors include, but are not limited to, Sirolimus (an immunosuppressant used to treat lymphangioleiomyomatosis, prevent organ transplant rejections, and treat perivascular epithelioid cell tumors, Everolimus (kinase inhibitor used to treat various types of malignancies), Temsirolimus (an antineoplastic agent used to treat renal cell carcinoma (RCC)), NVP-BEZ235 (dactolisib) (a generation DI compound developed by Novartis), OSI-027 (a potent and selective dual inhibitor of mTORC1 and mTORC2), Torin 1 (developed by AstraZeneca with a low nanomolar IC50 against mTOR), Ku-0063794 (an ATP-competitive mTOR inhibitor with strong anti-proliferative activity against cancer cells), AZD8055 (an orally accessible version of Ku-0063794 with antiproliferative action), and XL388 (a selective small-molecule ATP-competitive mTOR inhibitor that inhibits mTORC1 and mTORC2).

In some embodiments, the subject is a human patient suffering from, or suspected of suffering from, ccRCC.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Study design of Example 1.

FIG. 2. Evaluation and validation the prognostic value of CMB risk group (CMBRG). (A-C) in TCGA-KIRC cohort, CMBRG provides significant (A) and independent (B) patent stratification, and the combination of clinical factors and CMBRG significantly improve the prognostic power (C); (D-F) in CPTAC-CCRCC cohort, CMBRG provides significant (D) and independent (E) patent stratification, and the combination of clinical factors and CMBRG significantly improve the prognostic power (F); (G-I) in ZJU-CCRCC cohort, CMBRG provides significant (G) and independent (H) patent stratification, and the combination of clinical factors and CMBRG significantly improve the prognostic power (I). The p values were obtained using the log-rank test (A, D, G), the multivariate coxph regression (B, E, H) and the Mann-Whitney U test (C, F, I), respectively.

FIG. 3. The differences of molecular and clinical features between CMBRG Low and CMBRG High groups based on RNA-seq analysis in the TCGA-KIRC cohort. Specifically, mTOR, PPAR, and TGF-β are significantly enriched in the CMBRG Low group, while immune suppressive cells such as MDSC and Treg and IL6-JAK-STAT pathway are significantly enriched in the CMBRG High group.

FIG. 4. The differences of molecular and clinical features between CMBRG Low and CMBRG High groups based on RNA-seq analysis in the CPTAC-CCRCC cohort. Specifically, mTOR, PPAR, and TGF-β are significantly enriched in the CMBRG Low group, while immune suppressive cells such as MDSC and Treg and IL6-JAK-STAT pathway are significantly enriched in the CMBRG High group.

FIG. 5. The differences of molecular and clinical features between CMBRG Low and CMBRG High groups based on KEGG analysis on proteomics data in the CPTAC-CCRCC cohort. Pathways such as mTOR and HIF-1 are significantly enriched in the CMBRG Low group, while metabolic pathways such as Glycolysis/Gluconeogenesis and Fatty acid metabolism are significantly enriched in the CMBRG High group.

FIG. 6. Sensitivity prediction of mTOR inhibitors (i.e., rapamycin and temsirolimus) between CMBRG groups based on RNA-seq data using pRRophetic in TCGA-KIRC (A-B) and CPTAC-CCRCC (C-D) cohorts, where rapamycin/temsirolimus shows higher sensitivity in the CMBRG low group as indicated by lower predicted IC50 values.

FIG. 7. Validation of the predictive value of CMBRG on Everolimus response in ccRCC patients. (A) CMBRS is negatively and significantly associated with duration of Everolimus treatment, where the association and p value were obtained using the spearman correlation; (B) CMBRS is significantly different between responders and non-responders with Everolimus treatment, where the p value was obtained from the Mann-Whitney U test; (C) Everolimus treatment response rate is significantly higher in CMBRG low group than in CMBRG high group, where the p value was obtained from the Chi-square test.

FIG. 8. CMBRG is significantly associated with the CPTAC classification system. (A) In CPTAC-CCRCC cohort, proportion of CD8+ inflamed in the CMBRG High group is significantly higher than that in the CMBRG Low group, while VEGF immune desert is significantly lower in the CMBRG High group compared to the CMBRG Low group. (B) Leveraging the gene signatures from CPTAC-CCRCC immune subtypes, we explored the TCGA-KIRC cohort and observed similar distribution patterns of CD8+T inflamed and VEGF immune desert group.

FIG. 9. The profile of somatic mutations in the TCGA-KIRC cohort. Dysregulation of VHL was the most frequent alteration and was observed in 49% of tumors. PBRM1, TTN, SETD2 and BAP1 followed with mutation rates of 42%, 17%, 12%, and 11%, respectively.

FIG. 10. The profile of somatic mutations in the CPTAC-CCRCC cohort. Dysregulation of VHL was the most frequent alteration and was observed in 75% of tumors. PBRM1, BAP1, KDM5C and SETD2 followed with mutation rates of 44%, 15%, 15%, and 12%, respectively.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### AI-Empowered Cellular Morphometric Biomarkers Predict Treatment Response and Prognosis in Clear Cell Renal Cell Carcinoma Patients

Renal cell carcinoma (RCC), the most common type of kidney cancer, is a group of heterogeneous tumors originating in the nephron. The most common subtype of RCC is clear cell renal cell carcinoma (ccRCC), which accounts for approximately 75% of all RCC, and is a heterogeneous disease in the form of histopathology, molecular mechanism and clinical outcome. In this study, we developed a 73 cellular morphometric biomarker (CMB) signature, the corresponding CMB risk score (CMBRS) and CMB risk group (CMBRG) for the prediction of prognosis and treatment response in ccRCC patients. The prognostic and predictive value of CMBRS and CMBRG were validated in additional public and hospital cohorts. In addition, the molecular annotation of CMBRG using genomic and proteomic data provides the potential mechanism of the clinical significance of CMBRG. Our study establishes and validates an accurate and robust patient stratification with significant association with underly molecular and biological mechanisms and clinical outcomes in ccRCC, and warrants multicenter validation in our future study.

### Methods

### Retrospective Study Design

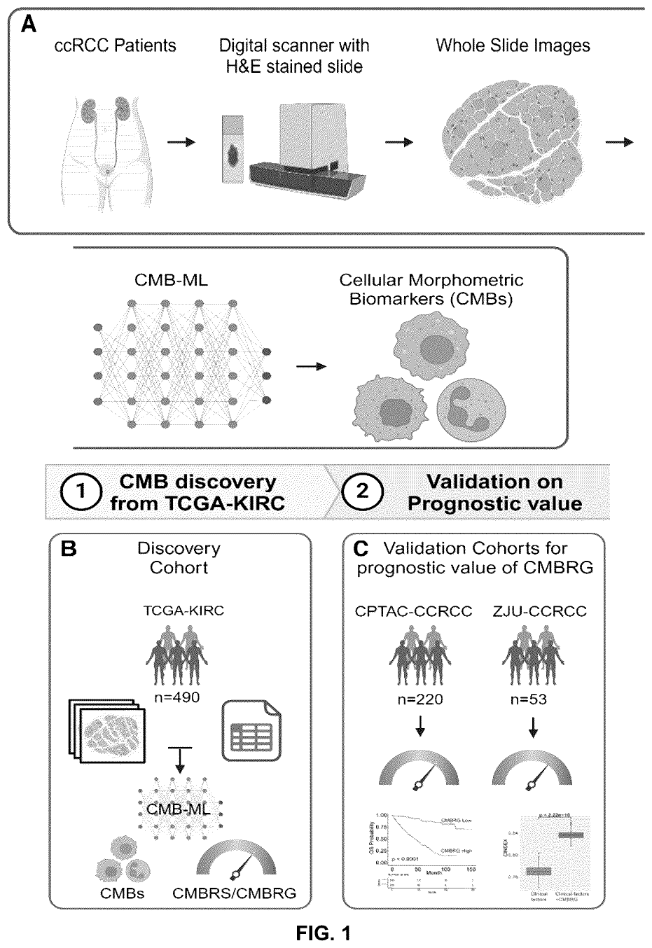

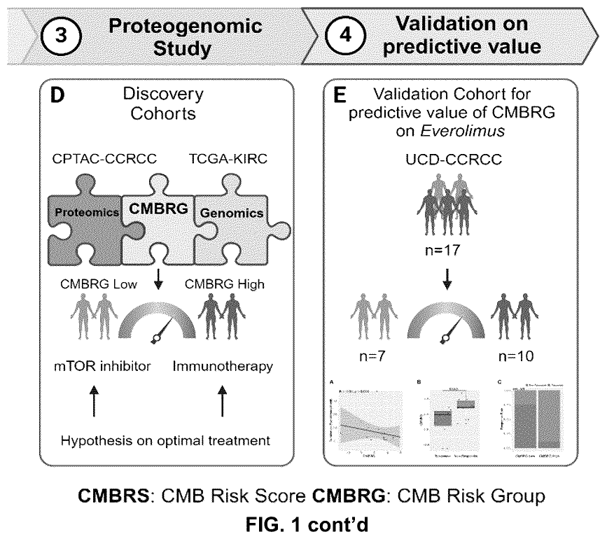

The study design was illustrated in FIG. 1. Specifically, the CMB-ML pipeline was applied on whole slide images (WSI) of diagnostic slides from 490 patients with both diagnostic slides and clinical data in the Cancer Genome Atlas Kidney Rental Clear Cell Carcinoma (TCGA-KIRC) project for CMB identification and model construction (FIG. 1, panel B). Three validation cohorts were used, including one public cohort (i.e., CPTAC-CCRCC [5, 6]; FIG. 1, panel C), and two independent hospital validation cohorts that were retrospectively collected at the Second Affiliated Hospital of Zhejiang University School of Medicine (ZJU-CCRCC, FIG. 1, panel C) and UC Davis (UCD-CCRCC, FIG. 1, panel E) for the validation of prognosis and the response to mTOR inhibitor, respectively. Specifically, the CPTAC-CCRCC cohort consists of 220 patients; and the UCD-CCRCC consisted of 17 patients; and the ZJU-CCRCC cohort consisted of 53 patients between 2015 and 2022. Hospital validation studies were independently carried out at collaborating hospitals, and were approved by the Ethics Board of the corresponding hospitals with a waiver of informed consent. Clinical-epidemiological and histopathological features are summarized in Supplementary Tables 1-4.

Identification of Cellular Morphometric Biomarkers (CMBs) and Construction of the CMB Risk Score (CMBRS) and CMB Risk Group (CMBRG) from the TCGA-KIRC Cohort

Based on the stacked predictive sparse decomposition (SPSD) [7] technique and our cellular morphometric biomarker via machine learning (CMB-ML) pipeline [4, 8, 9], we defined 256 CMBs from cellular objects extracted from the whole slide images (WSI) of H&E stained tissue histology sections from the diagnostic slides of 490 patients in the TCGA-KIRC project. In the CMB-ML pipeline, we used a single network layer with 256 dictionary elements (i.e., CMBs) and a sparsity constraint of 30 at a fixed random sampling rate of 1000 cellular objects per WSIs from the cohort. The pre-trained SPSD model reconstructed each cellular region as a sparse combination of pre-defined 256 CMBs and thereafter represents each patient as an aggregation of all delineated cellular objects belonging to the same patient.

The prognostic effect of high or low levels of each CMB on overall survival (OS) was assessed by Kaplan-Meier analysis (survminer package in R, version 0.4.8) and log-rank test (survival package in R, version 3.2-3), where the TCGA-KIRC cohort was divided into two groups (i.e., CMB-high and CMB-low groups) based on each CMB (cut-off estimated using survminer package in R, version 0.4.8). The set of CMBs as a prognostic signature was selected via a multivariate CoxPH regression model, including these CMBs with a significant effect on poor outcome events.

The construction of the CMB risk score (CMBRS) was defined below, where the coefficients of the final CMBs as categorical variables were obtained from multivariate CoxPH regression analysis:

$$CMBRS = {\sum }_{i = 1}^{N} ( coefficient of {CMB\_Category}^{i} ) * ( {CMB\_Category}^{i} )$$

Where N is the number of final CMBs that were independently and significantly associated with poor outcome events, and CMB_Categoryⁱ is the category of the i^{th} CMB (i.e., CMB-high=1; CMB-low=0). After CMBRS construction, the TCGA-KIRC cohort was divided into high-risk group and low-risk group based on CMBRS (cut-off estimated using survminer package in R, version 0.4.8). The CMBRS cut-off was then recorded and fixed for as the CMBRG model.

### Clinical Validation of CMBRS and CMBRG Models

The identical final CMBRS system, pre-established from the TCGA-KIRC cohort, was deployed to score the patients in the all three validation cohorts (i.e., CPTAC-CCRCC, ZJU-CCRCC and UCD-CCRCC), and the pre-established CMBRG (i.e., pre-established cut-off on CMBRS) was then applied on the CMBRS of each patient to assign the patient with risk level/group (i.e., high-/low-risk). The prognostic value of CMBRG was validated using CPTAC-CCRCC and ZJU-CCRCC cohorts, and the predictive value on mTOR inhibitor was evaluated using UCD-CCRCC cohort.

### Calculation of the TME-Related Signature Scores

After reviewing previously published studies, the Molecular Signatures Database (MSigDB; webpage for: gsea-msigdb.org/gsea/msigdb/index.jsp), and the Reactome pathway portal (webpage for: reactome.org/PathwayBrowser/), we identified relevant biomarker genes for tumor, immune, stromal, and metabolic reprogramming signatures. The 61 TME-related signature as well as the source of each signature was included in this study. GSVA with default parameters using the R package ‘GSVA’ was performed to calculate the signature score of each TME-related signature for each sample of each cohort separately. Besides, to reveal the tumor-immune interaction and identify the difference of TME between CMB high risk and low risk group, we analyzed the relevant molecules or pathways such as immune checkpoint genes, immune suppression signatures, immune exclusion signatures and immune exhaustion signatures by the “IOBR” R package. Except special indicating, the visualization of heatmap was achieved by using the R package ggplot2 (version 3.4.1).

### Calculation of the Other Biological Pathway Enrichment Scores

Human metabolism-related pathways were obtained from the KEGG database (webpage for: genome.jp/kegg/). The 86 human metabolism-related pathways and ten oncogenic signatures containing mTOR and HIFI signature were retrieved from a previously published study. GSVA was performed to calculate the enrichment score of each signature for each sample of each cohort separately based on the relative. To identify the potential differences in the biological functions of genes among high and low risk group, GSEA was performed based on the gene signatures using the R package ‘clusterprofiler’.

### RNA-Seq Data Analysis and Proteomics Analysis

Differentially expressed genes (DEGs) of mRNA and proteomics between high and low risk group were identified by applying the R package limma (version 3.5.1) with |log 2FC|>1 and adjusted P value <0.05. Simplify enrichment analysis based on DEGs between high and low risk group. Kyoto Encyclopedia of Genes and Genomes (KEGG) enrichment analyses of differential protein and gene set enrichment analysis (GSEA) of all Differential protein were conducted using the R package clusterProfiler (version 4.6.2). To better illustrate the differential enriched pathways among the high and low risk group, we performed gene set variation analysis for hallmark, GO and KEGG from MSigDB collections as well as tumor microenvironment score-related gene sets. We used the R package simplifyEnrichment (version 1.8.0) to slim down and visualize the results of KEGG function enrichment. All of the mass spectrometry data on TCGA tumor samples are deposited at the CPTAC Data Coordinating Center as complete protein assembly data sets for public access (webpage for: cptac-data-portal/).

### Construction of a Network Involving CMB and Gene Expression and Signatures

The above signatures were evaluated by Wilcoxon Rank Sum tests for significance between high and low risk group and then sent for CMBs correlation analysis using Pearson correlation analysis.

Evaluation of the Association Between CMBRG and the Sensitivity to mTOR Inhibitors Using RNA-Seq Data

We estimated the half-maximal inhibitory concentration (IC50) of mTOR related common chemotherapeutic agents (i.e., Rapamycin, and Temsirolimus) using the “pRRophetic” R package. Additionally, we screened the drug-target genes using the Drugbank database.

Validation of the Association Between CMBRG and the Sensitivity to mTOR Inhibitors in an Independent Hospital Cohort

An independent hospital cohort with 17 ccRCC patients treated with Everolimus was collected at UC Davis (UCD-CCRCC). The responders were defined as patients that stayed on Everolimus >3 months; and non-responders were defined as patients that stayed on Everolimus ≤3 months, where the 3-month-cutoff was determined based on the median progression-free survival (4-5 months) of ccRCC patients on Everolimus in the 3rd line and beyond setting [10-12].

### Statistical Analyses

The baseline characteristics and operative variables of patients were summarized using frequencies, percentages, mean±standard deviation, or median (range), depending on whether the variables were categorical or continuous. variables were compared using either Student's t-test, Mann-Whitney U test, or Kruskal-Wallis H test. Categorical variables were compared using either the x2 test with Yates correction or Fisher's exact test using the linear-by-linear association method. The Kaplan-Meier method with log-rank test was utilized to generate the survival curves. OS was defined as the time from surgery to death, while recurrence-free survival (RFS) was defined as the time from surgery to death or new tumor occurrence. Hazard ratios (HRs) and 95% confidence intervals (CIs) were reported. The statistical analyses were performed using R 4.2.2. Statistical significance was set at P<0.05, two-tailed.

### Results

CMBRG Is an Independent Predictor for Prognosis and Treatment Response in ccRCC Patients

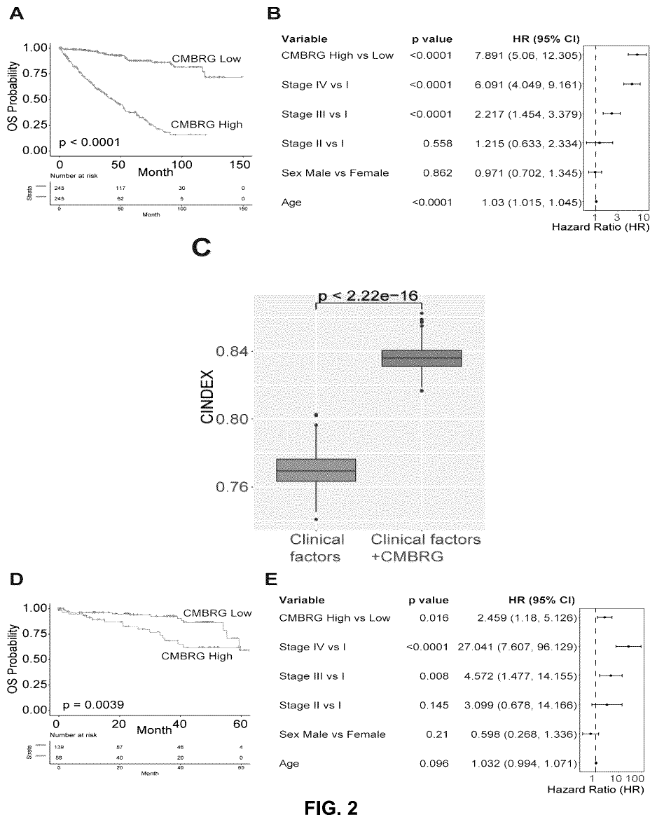

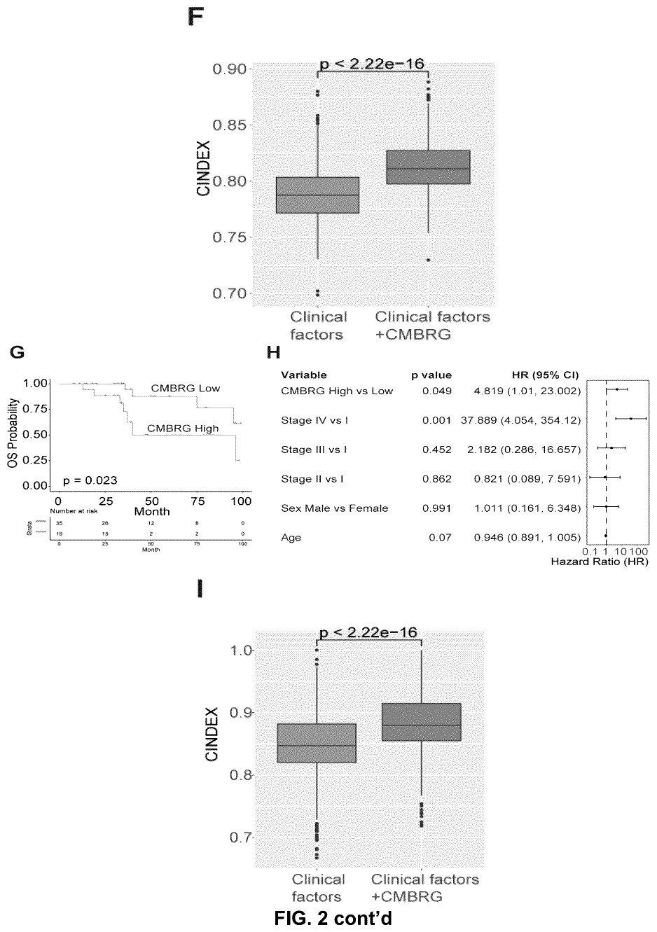

CMBRG demonstrates significant and independent prognostic value in all three ccRCC cohorts (FIG. 2). Specifically, CMBRG stratified ccRCC patients into low and high groups that have significant difference in overall survival (OS) in TCGA-KIRC (FIG. 2, panel A, p<0.0001), CPTAC-CCRCC (FIG. 2, panel D, p=0.0039) and ZJU-CCRCC (FIG. 2, panel G, p=0.023) cohorts. Importantly, CMBGR remains an independent and significant prognostic factor after adjusting clinical factors, including Stage, Age and Sex, in TCGA-KIRC (FIG. 2, panel B, p<0.0001; HR: 7.891, 95% CI [5.06, 12.305]), CPTAC-CCRCC (FIG. 2, panel E, p=0.016; HR: 2.459, 95% CI [1.18, 5.126]), and ZJU-CCRCC (FIG. 2, panel H, p=0.049; HR: 4.819, 95% CI [1.01, 23.002]) cohorts. Unsurprisingly, the combination of CMBRG with clinical factors significantly improve the prognostic power than only using clinical factors in TCGA-KIRC (FIG. 2, panel C, p<0.0001), CPTAC-CCRCC (FIG. 2, panel F, p<0.0001) and ZJU-CCRCC (FIG. 2, panel I, p<0.0001) cohorts.

### Signature Association Analysis (GSVA) Among CMBRG in TCGA-KIRC and CPTAC-CCRCC Cohorts (RNA-Seq)

The differences of molecular and clinical features between CMBRG low and CMBRG high groups are characterized by the tumor functional states features related signatures, as well as other TME-related signatures, including immune and stromal components, and metabolic reprogramming signatures.

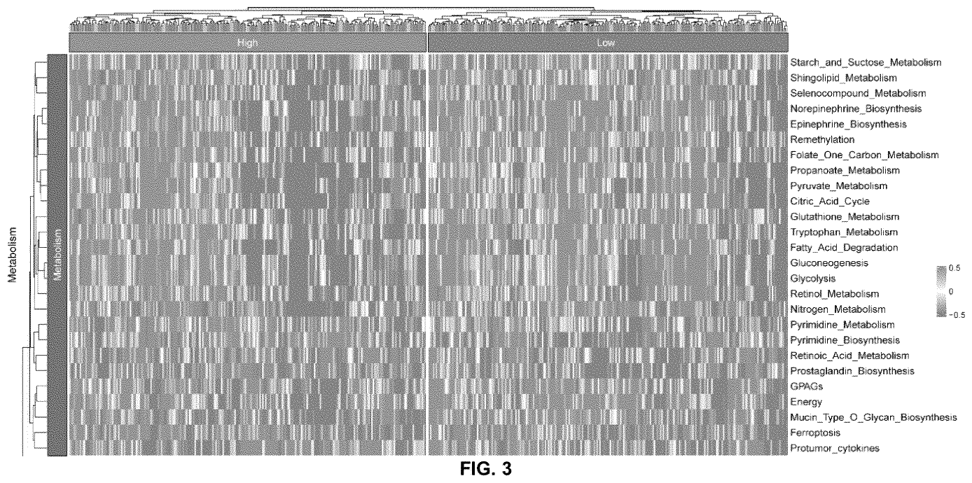

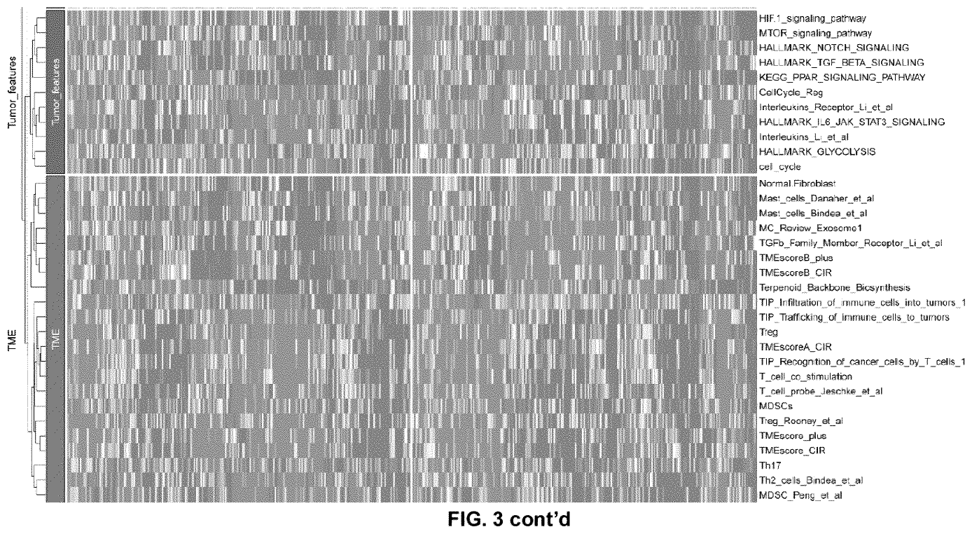

The analysis in TCGA-KIRC cohort using RNAseq data reveals significant differences in tumor features, tumor microenvironment (TME), and metabolism between two groups (FIG. 3). Specifically, pathways such as motor, PPAR, and TGF-β are significantly enriched in the low group, while immune suppressive cells such as MDSC and Treg and IL6-JAK-STAT pathway are significantly enriched in the high group. There are also substantial differences between the two groups in terms of metabolism.

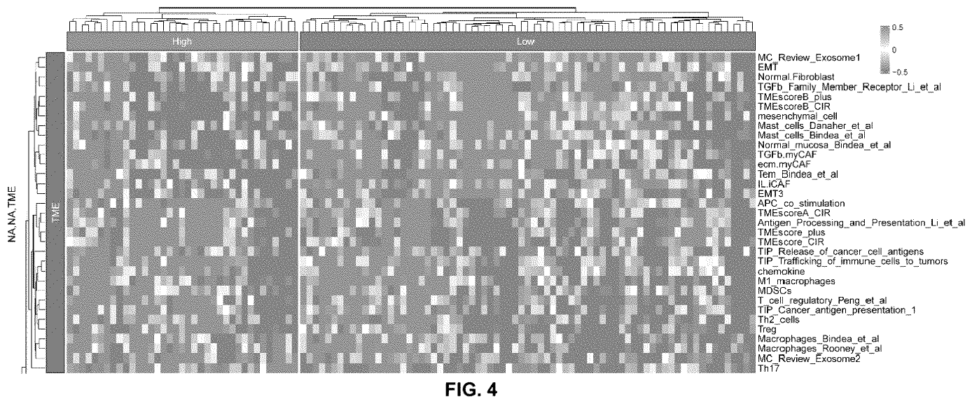

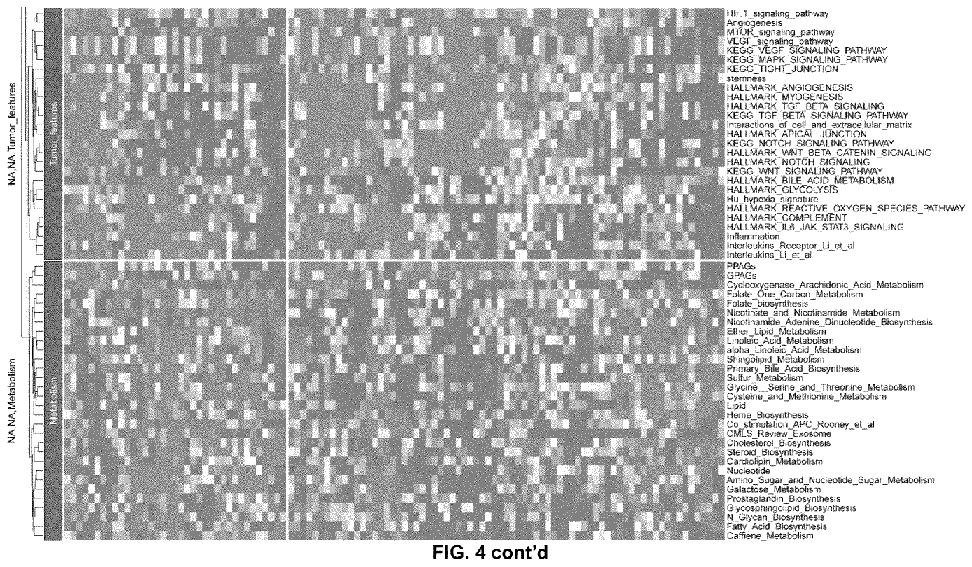

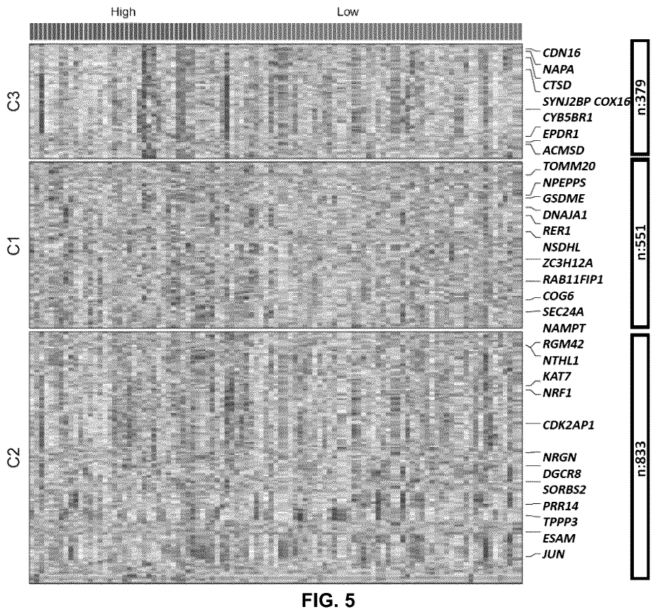

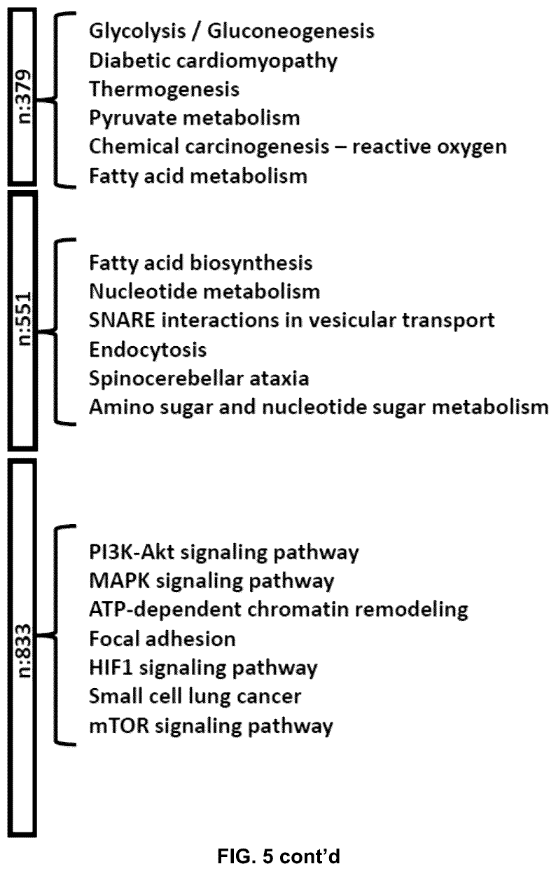

Similarly, the analysis in CPTAC-CCRCC cohort using RNA-seq data also revealed significant differences in tumor features, TME, and metabolism between two CMBRG groups (FIG. 4). Specifically, pathways such as mTOR, PPAR, and TGF-β are significantly enriched in the CMBRG low group, while immune suppressive cells such as MDSC, Treg and IL6-JAK-STAT pathway are significantly enriched in the CMBRG high group. There are also substantial differences between the two groups in terms of metabolism (FIG. 4). Furthermore, the KEGG analysis of proteomics data in the CPTAC-CCRCC cohort indicated that pathways such as mTOR and HIF-1 are significantly enriched in the CMBRG low group, while metabolic pathways such as Glycolysis/Gluconeogenesis and Fatty acid metabolism are enriched in the CMBRG high group (FIG. 5).

CMBRG is Significantly Associated with mTOR Inhibitor Sensitivity

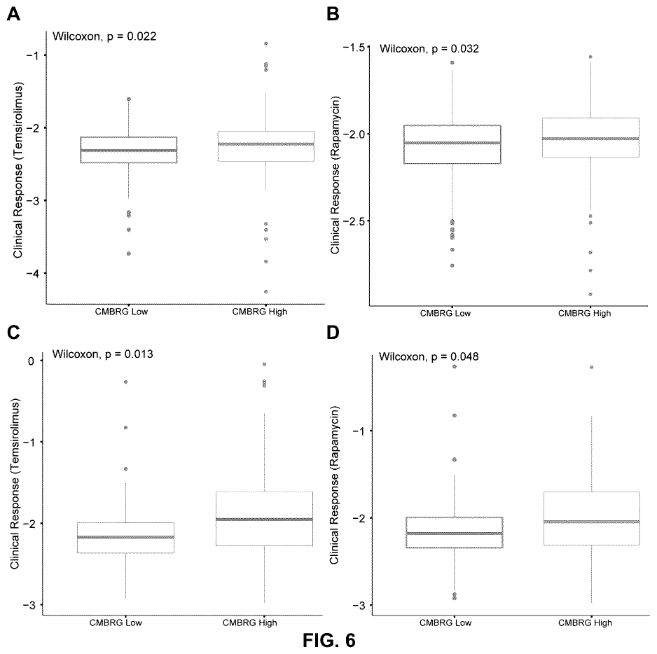

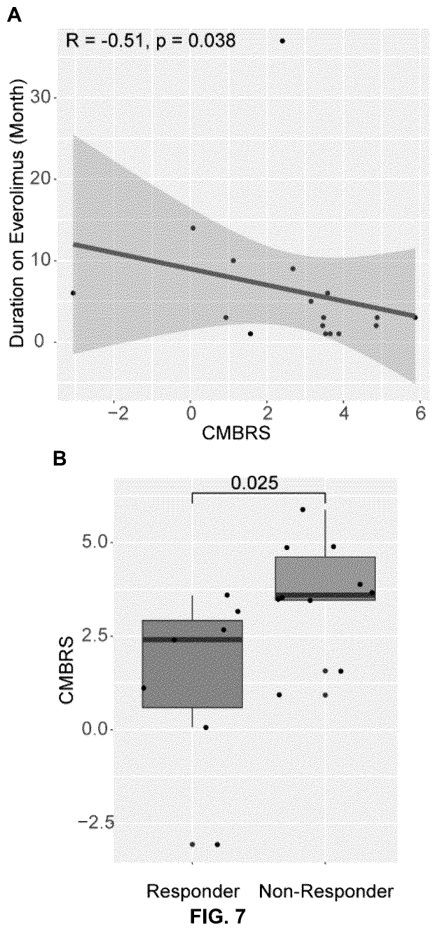

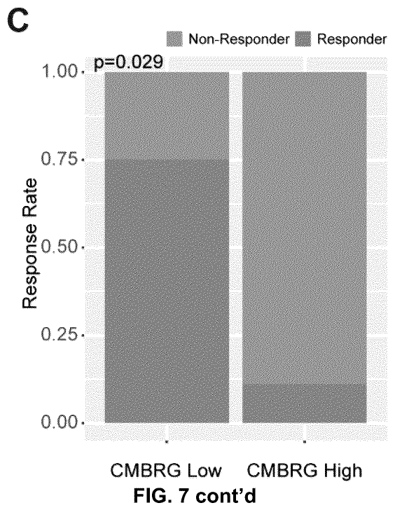

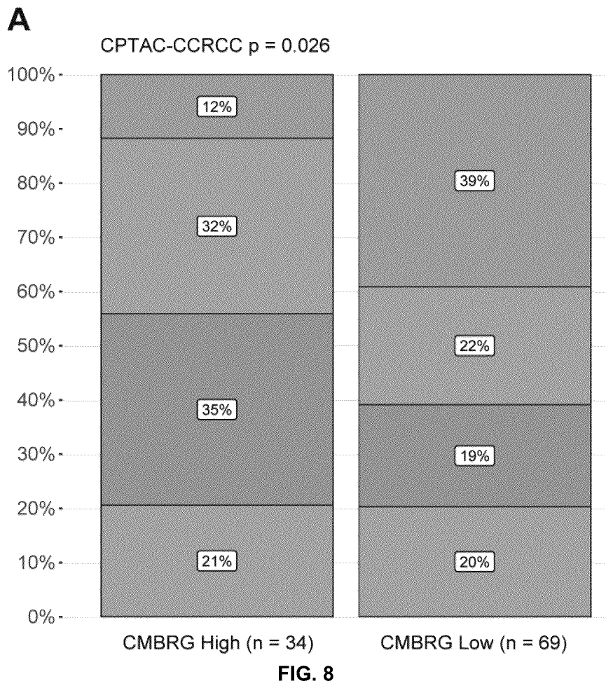

The significant differences in tumor features, TME, and metabolism between CMBRG groups suggest that ccRCC patients in CMBRG low and high groups may benefit from different treatment regimes. As a further evaluation, we estimated the sensitivity to mTOR inhibitors (i.e., rapamycin and temsirolimus) of each ccRCC patients using the RNA-seq data in both TCGA-KIRC and CPTAC-CCRCC cohorts. The results in both cohorts consistently suggested that patients in CMBRG low group are significantly more sensitive to mTOR inhibitors (i.e., significantly lower IC50 values in CMBRG low group than in CMBRG high group; FIG. 6). Exactingly, the significantly more sensitivity of ccRCC patients in CMBRG low group is validated in an independent hospital cohort (UCD-CCRCC) with 17 ccRCC patients (7 responders and 10 non-responders) treated with Everolimus. Specifically, CMBRS is significantly and negatively associated with treatment duration (FIG. 7, panel A, R=0.51, p=0.038), and is significantly different between responders and non-responders (FIG. 7, panel B, p=0.025). Importantly, patients in CMBRG low group are significantly more sensitive to mTOR inhibitor (i.e., Everolimus) than the ones in CMBRG high group (FIG. 7, panel C, p=0.029).

CMBRG is Significantly Associated with ccRCC Immune Subtypes

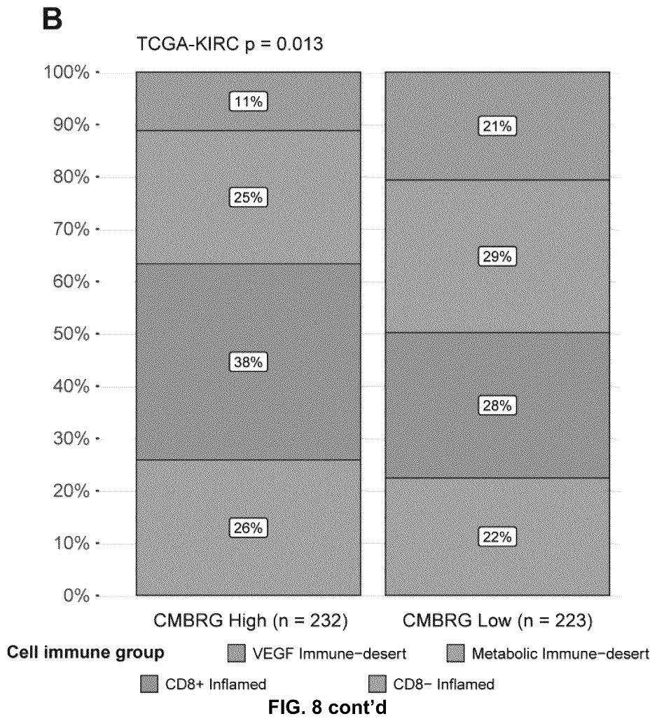

Adopting general features of immune-based groupings described previously and incorporating transcriptomic and proteomic features, CPTAC group defined four tumor subtypes in this ccRCC cohort: CD8+ inflamed, CD8− inflamed, VEGF immune desert, and metabolic immune desert. These subtypes were characterized by unique genomic alterations and TME signatures and discriminating signaling pathways that could be leveraged to predict therapeutic response. We compared our classification system with the CPTAC classification system and found that the proportion of CD8+ inflamed in the CMB high-risk group is significantly higher than that in the CMB low-risk group, while VEGF immune desert is significantly lower in the CMBRG high group compared to the CMBRG low group (chi-square test p=0.026) (FIG. 8, panel A). Leveraging the gene signatures from CPTAC immune subtypes, we explored the TCGA-KIRC cohort and observed similar distribution patterns of CD8+T inflamed and VEGF immune desert group (chi-square test p=0.013) (FIG. 8, panel B). The latter result reflects the aggregation of multiple features in the CD8+Inflamed subtype that are considered as poor prognosticators in ccRCC, including higher frequency of MDSC and Treg immune infiltration, increased proportion of higher-grade tumors, and inflammation TME. We did not detect an association of tumor mutational burden (TMB) or neoantigen load with any of CMB subtypes.

### Genomic Landscape of CMB High Risk and Low Risk Group

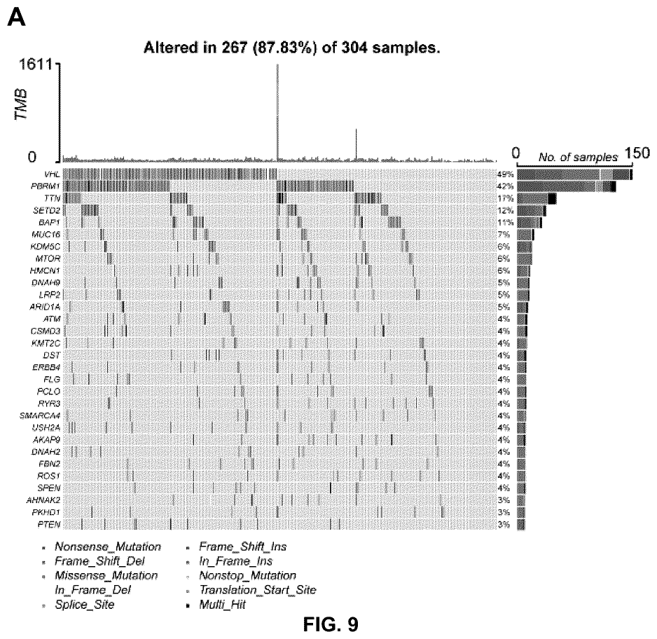

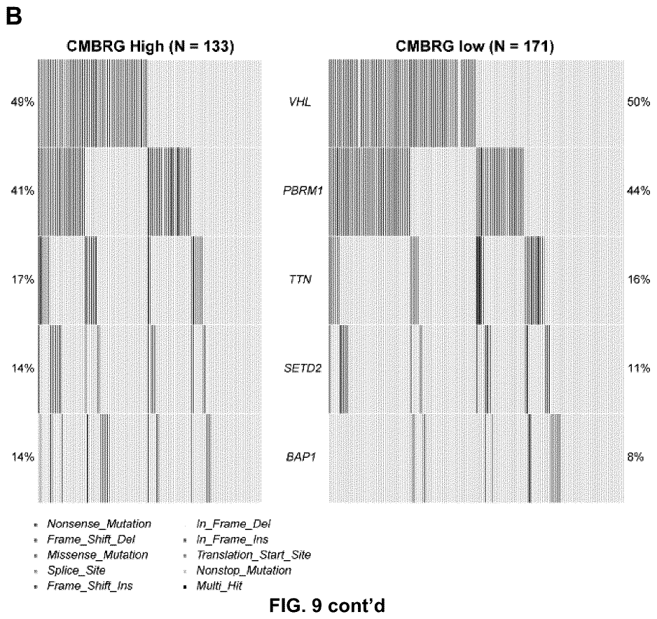

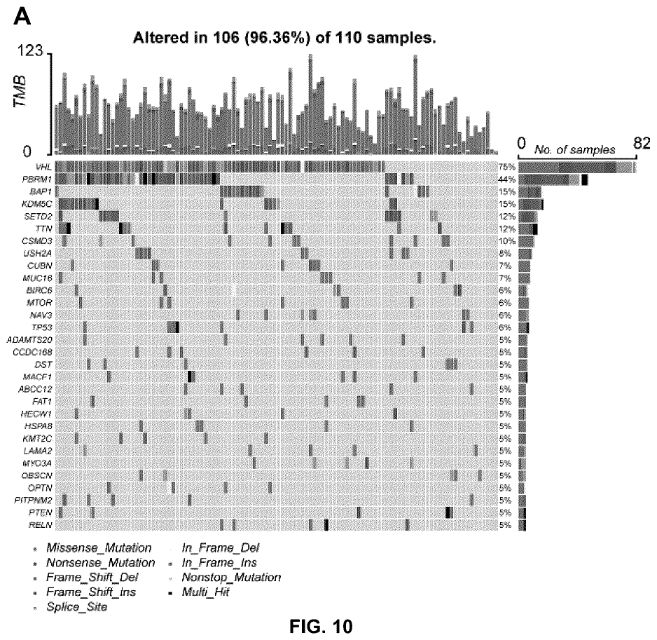

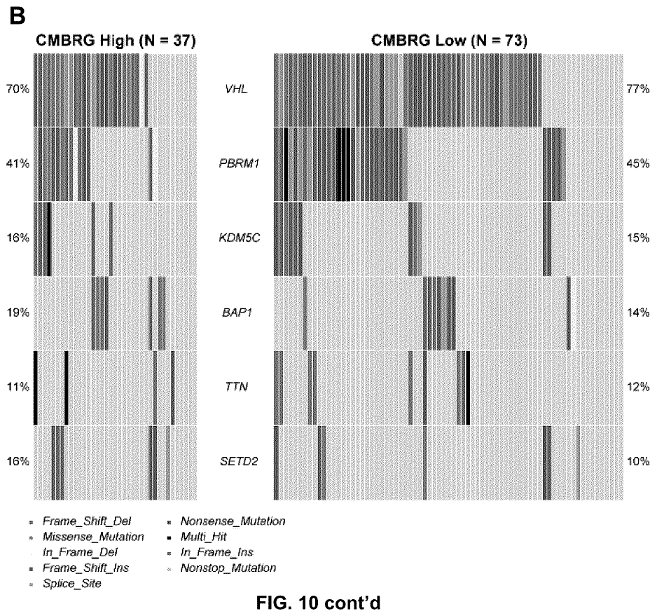

The profile of somatic mutations in the TCGA-KIRC cohort Dysregulation of VHL was the most frequent alteration and was observed in 49% of tumors. PBRM1, TTN, SETD2 and BAP1 followed with mutation rates of 42%, 17%, 12%, and 11%, respectively (FIG. 9). We indicate that these features do not differ in CMBRG high and low groups. The profile of somatic mutations in the CPTAC-CCRCC cohort Dysregulation of VHL was the most frequent alteration and was observed in 75% of tumors. PBRM1, BAP1, KDM5C and SETD2 followed with mutation rates of 44%, 15%, 15%, and 12%, respectively (FIG. 10). We indicate that these features do not differ in CMBRG high and low groups.

### Discussion

The targeted therapy for advanced renal cell carcinoma comprises two distinct categories of mechanisms, yet they exhibit some functional overlap. Current Frontline therapy therapies for advanced ccRCC target VEGF and mTOR. Bevacizumab, sunitinib, and sorafenib act by inhibiting the vascular endothelial growth factor (VEGF) pathway, which is a critical mediator of tumor angiogenesis, along with direct anti-tumor activity. In contrast, temsirolimus and everolimus act by specifically inhibiting the mammalian target of rapamycin (mTOR) kinase signaling pathway, resulting in cell cycle arrest, enhanced apoptosis, and suppression of angiogenesis. Both the VEGF and mTOR pathways are upregulated by intrinsic factors in tumors, such as hypoxia, cytokine release, inactivation of oncogenes, and tumor suppressor genes. While both pathways enhance tumor angiogenesis, their targeted inhibitors demonstrate different anti-tumor activities and side effects [13]. We hypothesize that these patients in CMBRG low group should be treated with anti-VEGF and mTOR inhibitors.

Tyrosine Kinase Inhibitors (TKI) has a minimal impact on T cell activity and can improve the tumor microenvironment by downregulating STAT3 expression and reversing immune suppression mediated by myeloid-derived suppressor cells (MDSCs) and regulatory T cells (Tregs) [14]. It enhances natural killer (NK) cell recognition, increases recruitment and infiltration of CD4+ T cells and CD8+ T cells, among other pathways, thereby enhancing the efficacy of immunotherapy. In mouse models, blocking IL-6 is associated with improved tumor control and higher densities of CD4 and CD8 effector T cells. Additionally, combined therapy of blocking IL-6 and CTLA-4 monoclonal antibody promotes increased tumor infiltration of CD8 T cells, while conversely reducing Treg, Th17, MDSCs, and macrophages, resulting in increased ratios of CD8:Treg and CD8:MDSC. Thus, blocking IL-6 can enhance the therapeutic efficacy of immune checkpoint inhibitors by improving the tumor immune microenvironment [14]. We hypothesize that these patients in CMBRG high group should be treated combined with anti-VEGF, immune checkpoint inhibitors with interleukin-6 blockade.

Patient stratification based on the CPTAC immune four subtypes revealed that VEGF immune desert tumors were associated with improved patient survival, while CD8+Inflamed tumors were associated with poor patient outcome [5]. This can be mutually validated with our results. CMBRG high group with more frequence of VEGF immune desert deserved a worse prognosis compared with CMBRG low group with more frequence of CD8+Inflamed tumors.

### Conclusions

This study suggests that CMBRG provide additional prognostic and predictive value in ccRCC patients beyond the limits of solely classic clinical and histopathological risk features. Multicenter validation studies with large cohorts are needed to assess the role of CMBRG in clinical practice.

### REFERENCES CITED HEREIN

1. Pedrosa, I., et al., MR imaging of renal masses: correlation with findings at surgery and pathologic analysis. Radiographics, 2008. 28(4): p. 985-1003.
2. Prasad, S. R., et al., Common and Uncommon Histologic Subtypes of Renal Cell Carcinoma: Imaging Spectrum with Pathologic Correlation. RadioGraphics, 2006. 26(6): p. 1795-1806.
3. Creighton, C. J., et al., Comprehensive molecular characterization of clear cell renal cell carcinoma. Nature, 2013. 499(7456): p. 43-49.
4. Liu, X.-P., et al., Clinical significance and molecular annotation of cellular morphometric subtypes in lower-grade gliomas discovered by machine learning. Neuro-Oncology, 2022. 25(1): p. 68-81.
5. Clark, D. J., et al., Integrated Proteogenomic Characterization of Clear Cell Renal Cell Carcinoma. Cell, 2019. 179(4): p. 964-983.e31.
6. (CPTAC), N.C.I.C.P.T.A.C., The Clinical Proteomic Tumor Analysis Consortium Clear Cell Renal Cell Carcinoma Collection (CPTAC-CCRCC) (Version 13) [Data set]. 2018, The Cancer Imaging Archive.
7. Chang, H., et al., Stacked Predictive Sparse Decomposition for Classification of Histology Sections. Int J Comput Vis, 2015. 113(1): p. 3-18.
8. Chang, H., et al., From Mouse to Human: Cellular Morphometric Subtype Learned From Mouse Mammary Tumors Provides Prognostic Value in Human Breast Cancer. Frontiers in Oncology, 2022. 11.
9. Mao, X. Y., et al., iCEMIGE: Integration of CEll-morphometrics, MIcrobiome, and GEne biomarker signatures for risk stratification in breast cancers. World J Clin Oncol, 2022. 13(7): p. 616-629.
10. Gulati, S. and P. N. Lara, Belzutifan versus Everolimus in Advanced Kidney Cancer: A Commentary on LITESPARK-005 Trial from ESMO 2023. Kidney Cancer, 2024. 8(1): p. 23-24.
11. Albiges, L., et al., LBA88 Belzutifan versus everolimus in participants (pts) with previously treated advanced clear cell renal cell carcinoma (ccRCC): Randomized open-label phase III LITESPARK-005 study. Annals of Oncology, 2023. 34: p. S1329-S1330.
12. Motzer, R. J., et al., Nivolumab versus Everolimus in Advanced Renal-Cell Carcinoma. New England Journal of Medicine, 2015. 373(19): p. 1803-1813.
13. Posadas, E. M., S. Limvorasak, and R. A. Figlin, Targeted therapies for renal cell carcinoma. Nat Rev Nephrol, 2017. 13(8): p. 496-511.
14. Ozao-Choy, J., et al., The novel role of tyrosine kinase inhibitor in the reversal of immune suppression and modulation of tumor microenvironment for immune-based cancer therapies. Cancer Res, 2009. 69(6): p. 2514-22.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for predicting whether a subject with clear cell renal cell carcinoma (ccRCC) will be responsive to targeted therapy or immunotherapy will be efficacious for treating a subject with ccRCC, comprising: (a) obtaining a ccRCC tumor sample from a subject suffering from ccRCC, or suspected thereof; (b) determining or measuring the value or quantity of one or more cellular morphometric biomarkers (CMB); (c) correlating the value or quantity determined or measured for the CMBs to determine whether the subject would benefit from a targeted therapy or immunotherapy; and (d) treating the subject with the targeted therapy or immunotherapy.

2. The method of claim 1, wherein the targeted therapy comprises administering a tyrosine kinase inhibitor (TKI), mTOR inhibitor, immune checkpoint inhibitors, or a combination therapy to the subject.

3. The method of claim 2, wherein the tyrosine kinase inhibitor (TKI) is sunitinib, pazopanib, axitinib, or cabozantinib.

4. The method of claim 2, wherein the mTOR inhibitor is everolimus or temsirolimus.

5. The method of claim 2, wherein the immune checkpoint inhibitor is Pembrolizumab, nivolumab, or atezolizumab.

6. The method of claim 2, wherein the combination therapy comprises administering nivolumab plus ipilimumab, or pembrolizumab plus axitinib, to the subject.

7. The method of claim 1, wherein the immunotherapy therapy is a cytokine therapy or immune checkpoint inhibitor therapy.

8. The method of claim 7, wherein the immune checkpoint inhibitor therapy comprises administering an pembrolizumab, nivolumab, and/or atezolizumab to the subject.

9. A method for predicting whether a subject with clear cell renal cell carcinoma (ccRCC) will be responsive to targeted therapy or immunotherapy will be efficacious for treating a subject with ccRCC, comprising: (a) obtaining a ccRCC tumor sample from a subject suffering from ccRCC, or suspected thereof; (b) determining or measuring the value or quantity of one or more cellular morphometric biomarkers (CMB); (c) calculating a Cellular Morphometric Biomarker Risk Score (CMBRS) using the CMBs determined or measured in step (b) using the formula:
$$CMBRS = {\sum }_{i = 1}^{N} ( coefficient of {CMB\_Category}^{i} ) * ( {CMB\_Category}^{i} ) ;$$
(d) correlating the CMBRS to a corresponding CMB risk group (CMBRG) to identity whether the subject is of a CMBRG low group or CMBRG high group; and (e) treating the subject wherein a patient belonging to the CMBRG low group is treated with targeted therapy, while a patient belonging to the CMBRG high group is treated with immunotherapy.

10. The method of claim 9, wherein the CMBRS is calculated using 73 cellular morphometric biomarker (CMB) signature.

11. The method of claim 9, wherein the targeted therapy comprises administering a tyrosine kinase inhibitor (TKI), mTOR inhibitor, immune checkpoint inhibitors, or a combination therapy to the subject.

12. The method of claim 11, wherein the tyrosine kinase inhibitor (TKI) is sunitinib, pazopanib, axitinib, or cabozantinib.

13. The method of claim 11, wherein the mTOR inhibitor is everolimus or temsirolimus.

14. The method of claim 11, wherein the immune checkpoint inhibitor is Pembrolizumab, nivolumab, or atezolizumab.

15. The method of claim 11, wherein the combination therapy comprises administering nivolumab plus ipilimumab, or pembrolizumab plus axitinib, to the subject.

16. The method of claim 9, wherein the immunotherapy therapy is a cytokine therapy or immune checkpoint inhibitor therapy.

17. The method of claim 16, wherein the immune checkpoint inhibitor therapy comprises administering an pembrolizumab, nivolumab, and/or atezolizumab to the subject.
